(19) United States
(12) Patent Application Publication
(10) Pub. No.: US 2024/0252709 A1
(43) Pub. Date: Aug. 1, 2024

(54) Device for Treating and Sanitizing Air in an Inhabited Environment

(71) Applicant: Naxos Finance SA, Luxembouorg (LU)

(72) Inventor: Luigi Alluto, Moncalieri, Turin (IT)

(21) Appl. No.: 18/561,576

(22) PCT Filed: May 20, 2021
(86) PCT No.: PCT/IB2021/054380

§ 371 Date: Nov. 16, 2023

(51) Int. Cl. A61L 9/12; A61L 9/20

(52) U.S. Cl. CPC A61L 9/122; A61L 9/20; A61L 2209/111; A61L 2209/212

(57) ABSTRACT

A device for treating air in an inhabited environment, wherein the device includes a hollow body having a first opening for the entrance of an air flow and a second opening for the exit of the air flow, an air flow generator configured for producing the air flow within the hollow body, an ozone generator, a reaction chamber positioned inside the hollow body downstream of the ozone generator, wherein the reaction chamber is configured for changing one or more physical characteristics of the air flow within the hollow body, and an abating chamber configured for abating the residual ozone contained in the air flow exiting the reaction chamber.

### BACKGROUND OF THE INVENTION

### 1. The Field of the Invention

The present invention relates to a device for treating air in an inhabited environment; in particular, a device for sanitizing air in an inhabited environment by means of ozone.

### 2. The Relevant Technology

As is known, the wide circulation of pathogenic elements such as, for example, the SARS-CoV-2 virus, Candida auris spores or mycelium, or the like, has required the use of devices for treating and sanitizing the air in inhabited environments such as, for example, houses, workplaces, schools, buses, railway carriages, airplane cabins, etc.

In such places, air treating and sanitizing devices using ozone for oxidizing and destroying pathogenic elements can hardly be employed, since international safety regulations require that ozone concentration within inhabited environment should not exceed 0.05-0.1 ppm (parts per million).

This strongly limits the use of ozone in such environments, since ozone can sanitize in a satisfactory manner the air in an environment (by oxidizing any pathogens that may be present in the air) and the surfaces thereof only when it is available in a concentration in excess of 1-2 ppm. For this reason, ozone-based sanitization is only possible in uninhabited places, when no people are present.

For example, Chinese publication No. CN205316558U by CHANGTAI ZHENGLIANG LAMPS BUSINESS DEPT. describes a ceiling fan comprising an ozone generator, wherein said ozone generator is controlled by a controller connected to a sensor and configured for stopping the ozone generator when it detects, via said sensor, the presence of people in the room where said fan has been installed.

For this reason, air treating devices are employed in inhabited environments which internally contain ultraviolet (UV) lamps, activated charcoal filters, or the like, which however are not as effective as ozone.

### SUMMARY OF THE INVENTION

The present invention aims at solving these and others problems by providing a device for treating and sanitizing air in a domestic environment as set forth in the appended claims.

The basic idea of the present invention is to use, in the device for treating and sanitizing air in an inhabited environment, an ozone generator combined with a reaction chamber and ozone abating means. In said device, the reaction chamber is positioned downstream of an ozone generator, with reference to the air flow to be sanitized, and is configured (e.g., through proper shaping) to change one or more physical characteristics (e.g., pressure, temperature, enthalpy, velocity, turbulence, dew point, or the like) of said air flowing through the device, wherein said abating means are configured for abating the residual ozone concentration still contained in the air flow before the latter exits the device.

One of the most important functions of the reaction chamber is the generation of a turbulent motion of the air in the reaction chamber. This increases the probability that the ozone will interact with the air inside the device, thereby accelerating the air sanitization process while at the same time causing most of the ozone abatement process, which acts upon the ozone produced by the ozone generator, to occur within the air treating and sanitizing device.

It is thus possible to increase the quantity of ozone used for sanitizing the air in an inhabited environment, without dispersing ozone into such environment.

Further advantageous features of the present invention will be set out in the appended claims.

### BRIEF DESCRIPTION OF THE DRAWINGS

These features as well as further advantages of the present invention will become more apparent in the light of the following description of a preferred embodiment thereof as shown in the annexed drawings, which are provided merely by way of non-limiting example, wherein:

FIG. 1 shows a vertical sectional view of a first embodiment of an air treating and sanitizing device according to the invention;

FIG. 2 shows a graph representing the operation of the device of FIG. 1;

FIG. 3 shows a vertical sectional view of a second embodiment of an air treating and sanitizing device according to the invention;

FIG. 4 shows a vertical sectional view of a third embodiment of an air treating and sanitizing device according to the invention;

FIG. 5 shows a vertical sectional view of a fourth embodiment of an air treating and sanitizing device according to the invention;

FIG. 6 shows a vertical sectional view of a fifth embodiment of an air treating and sanitizing device according to the invention;

FIG. 7 shows a vertical sectional view of a sixth embodiment of an air treating and sanitizing device according to the invention;

FIG. 8 shows a vertical sectional view of a seventh embodiment of an air treating and sanitizing device according to the invention;

FIG. 9 shows a vertical sectional view of an eighth embodiment of an air treating and sanitizing device according to the invention;

FIG. 10 shows a vertical sectional view of a ninth embodiment of an air treating and sanitizing device according to the invention.

### DETAILED DESCRIPTION OF THE PREFERRED EMBODIMENTS

In this description, any reference to “an embodiment” will indicate that a particular configuration, structure or feature is comprised in at least one embodiment of the invention. Therefore, expressions such as “in an embodiment” and the like, which can be found in different parts of this description, will not necessarily refer to the same embodiment. Moreover, any particular configuration, structure or feature may be combined as deemed appropriate in one or more embodiments. The references below are therefore used only for simplicity's sake, and shall not limit the protection scope or extension of the various embodiments.

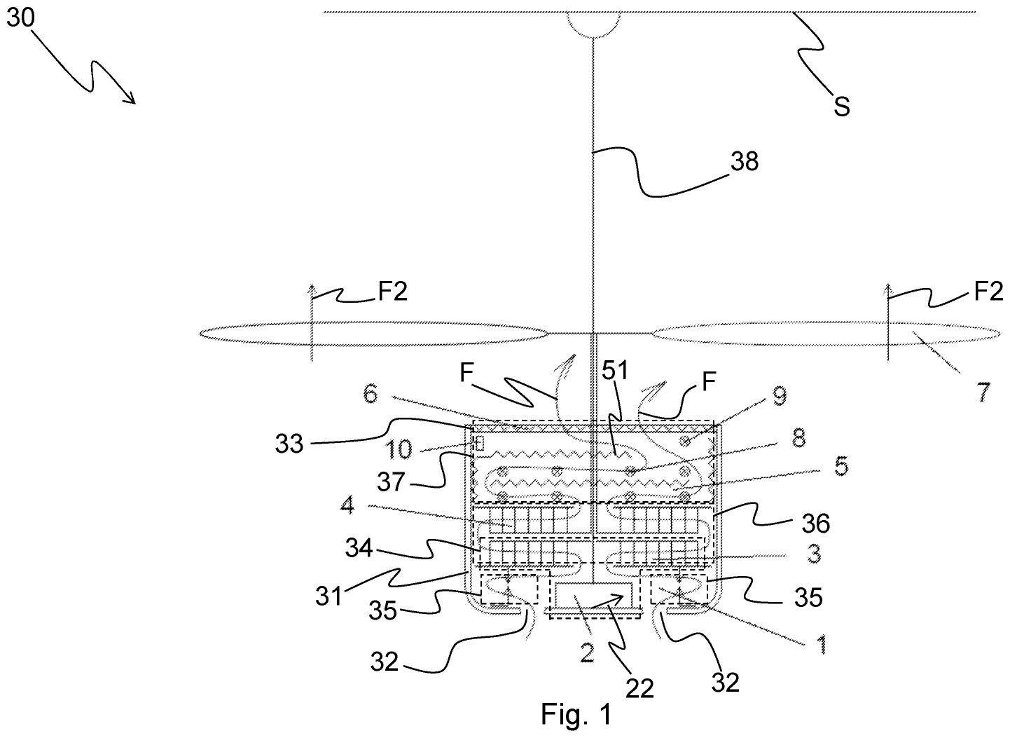

With reference to FIG. 1, the following will describe a first embodiment of a device 30 for treating and sanitizing air in an inhabited environment; such device 30 comprises the following elements:
a hollow body 31 (e.g., a boxed body) comprising a first opening 32 for the entrance of an air flow F and a second opening 33 for the exit of said air flow F;
an air flow generator 34 configured for producing said air flow F within said hollow body 31;
an ozone generator 35 configured for adding ozone to said air flow within said hollow body 31;
a reaction chamber 36 positioned within said hollow body downstream of the ozone generator 35, with reference to the air flow, wherein said reaction chamber is configured (e.g., through appropriate shaping) for changing one or more physical characteristics of the air flow F (e.g., pressure, temperature, enthalpy, velocity, turbulence, dew point, or the like) within said hollow body 31, i.e., said reaction chamber 36 preferably comprises means for compressing and expanding said air flow F within said hollow body (e.g., an intake rotor 3 and/or a turbine 4, which will be described below);
abating means 37 configured for abating the ozone concentration in the air flow F exiting said reaction chamber.

The device 30 preferably has a chandelier-like shape and preferably comprises a fixing cable or rod 38 through which it can be secured to a ceiling S.

Preferably, the hollow body 31 has a cylindrical or rectangular shape, with the first opening 32 positioned on the bottom surface of said hollow body 31 and having a ring-like shape, while the second opening 33 preferably occupies the entire top portion of said hollow body 31, i.e., the hollow body 31 is essentially open at the top.

The air flow generator 34 preferably comprises the following elements:
an intake rotor 3 which, as it rotates, can generate the air flow F entering through the first opening 32 and exiting through said second opening 33;
an electric motor 2, preferably of the induction or permanent-magnet type, coupled to the shaft of said intake rotor 3;
a speed controller 22 connected to the electric motor 2 and configured for controlling the revolution speed of said electric motor 2, and hence that of the intake rotor 3, so as to control/change the rate of flow F.

The ozone generator 35 preferably comprises an ozone production chamber positioned immediately downstream of the first opening 32 and crossed by the air flow F, wherein rotating tips (preferably integral with the lower portion of the intake rotor 3) are preferably positioned inside such production chamber, which are supplied with high voltage and generate sufficient ionization conditions for ozone to form within the air flow F.

It must be pointed out that any other type of ozone generator may be used (e.g., a glass tube comprising a pair of electrodes supplied with high voltage, or the like), without however departing from the teachings of the present invention.

The reaction chamber 36, which is positioned downstream of the ozone generator 35, preferably comprises a turbine 4 configured to pick up part of the energy contained in the air flow F (i.e., configured to be driven by said air flow F) generated by the air flow generator 34 and to transfer said energy to its own shaft; to this end, the device 30 preferably comprises a propeller 7 coupled to said turbine 4 and positioned outside the hollow body 31 to perform functions that will be described below in detail. In this way, pressure differences are generated along the path of the air flow, which make it advantageously turbulent, thereby speeding up the ozone reaction and hence its effectiveness in the process of sanitizing the air flow F.

The abating means 37 preferably comprise a chamber 5 that comprises mirrors 51 capable of reflecting C-type ultraviolet (UVC) light and UVC lamps 8,9, preferably having a wavelength of 254 nm, and more preferably of the LED type. The presence of luminous radiations in the UVC spectrum accelerates ozone decay, advantageously resulting in less ozone being dispersed in the environment. Moreover, the assembly consisting of the mirrors 51 and the UVC lamps 8,9 performs a dual function, i.e., to abate a quantity of ozone contained in the air flow F and also to sanitize the air by killing any pathogenic agents by emitting radiations in the UVC spectrum.

Furthermore, the abating means 37 comprise also a catalytic filter 6 (also known as “scrubber”) made of a material suitable to cause the interaction of any ozone (thus definitively abating it) that may still be present in the air flow F before it exits through the second opening 33, such as, for example, the filter used for “CDA Ozone scrubber” equipment; for this purpose, said catalytic filter 6 is preferably made of manganese dioxide and copper oxide, e.g., the material called Carulite® 200, or titanium dioxide, which can also be applied like a paint to the inside the chamber 37 and to the catalytic filter 6, which are located inside the hollow body 31.

It must be pointed out that titanium dioxide, when irradiated with ultraviolet light, generates a photocatalytic action that ensures a very effective abatement of the ozone (and also of any pathogens still suspended in the air flow F).

The catalytic filter is preferably positioned around or within the second opening 33, so that it can be replaced easily.

When the device 30 is in an operating condition, power is supplied to the speed controller 22 in such a way that it generates an electric current capable of rotating the electric motor 2, which in turn rotatably drives the intake rotor 3, thus generating the air flow F. After passing through the first inlet opening 32, this air flow F is enriched with ozone produced by the ozone generator 35 (which also receives an electric current), which preferably generates electric discharges caused by metal tips rotating integrally with the intake rotor 3. Subsequently, the ozone-enriched air, as it enters the reaction chamber 36, is fed into the turbine 4, thus undergoing considerable mixing and expansion, resulting in increased volume and velocity and lower pressure and temperature, thereby producing more frequent interactions between ozone molecules and organic molecules (including viruses, bacteria, fungi, spores) that may be present in the air flow F, occurring at a lower temperature due to isentropic expansion (that produces mechanical work which can be used to drive the propeller 7), preferably close to the dew point. This process produces fast ozone decay. The air flow F is then subjected to the action of the abating means 37, i.e., said flow enters the chamber 5, where it is irradiated with C-type ultraviolet light generated by the UVC lamps 8,9, which also receive an electric current, and which accelerate the ozone decay process (as it occurs in nature in the ozone layer above the earth). It should also be noted that the 254 nm UVC radiation contributes to sanitizing the air flow by destroying and/or making inert any pathogenic agents such as viruses (e.g., SARS-CoV-2 or other virus types), spores and bacteria, i.e., by damaging them to the point that they can no longer infect a person or a domestic animal.

It is thus possible to increase the quantity of ozone used for sanitizing an inhabited environment, without producing harmful ozone concentrations in such environment.

As aforementioned, the device 30 may comprise a propeller 7 coupled to the turbine 4 or to another drive system. The blades of this propeller are so oriented that, when said propeller is rotating, a second air flow F2 is generated, whose direction is (initially) parallel to the axis of the fixing cable or rod 38, and whose sense is such that said second air flow F2 will move away from the device 30, i.e., towards the ceiling S, thus incorporating the air flow F. This makes it possible to disperse upwards the air flow F exiting through the second opening, which may still contain some ozone and therefore would tend, because of its higher specific weight, to go down, where people and/or domestic animals may inhale it. By blowing the flow F up towards the ceiling S, the decay of any ozone still present downstream of the abating means 37 is accelerated because the air flow F2, as it meets the ceiling S, is slowed down and diverted; moreover, this feature permits reducing the formation of moulds on the ceiling S and on the surrounding walls. This makes it possible to prevent the inhabitants of an indoor environment from inhaling ozone, while the quantity of ozone used for sanitizing said environment can be increased without producing harmful ozone concentrations therein.

In addition to the above, the mirrors 51 are arranged in the chamber 5 in such a way as to extend the path of the flow F within the device 30 and reflect the light of the UVC lamps, thus advantageously increasing the time of exposure of the air flow F to the UVC radiations emitted by the UVC lamps 8,9 and reflected by said mirrors 51. This results in increased ozone abating power, obtained through the combined use of the mirrors 51 and the lamps UVC 8,9.

By improving the capability of abating ozone within the chamber 5, it is possible to increase the quantity of ozone used for sanitizing an inhabited environment, without producing harmful ozone concentrations therein.

It must be pointed out that the presence of the reaction chamber 36 upstream of the abating means 37 (in particular, upstream of the catalytic filter 6) advantageously reduces the quantity of ozone that said catalytic filter 6 has to cause to interact, thus extending its service life or, the quantity of ozone to be made to interact being equal, advantageously permitting a higher ozone concentration in the reaction chamber 36, thereby producing a greater air sanitizing effect.

It is thus possible to increase the quantity of ozone used for sanitizing an inhabited environment, without producing harmful ozone concentrations therein.

In addition to the above, the device 30 comprises also a sensor 10 positioned upstream of the catalytic filter 6 (i.e., in the chamber 5) and capable of producing a signal representing an ozone concentration in the chamber 5, and a control unit (not shown in the drawings), e.g., an ESP32 or ESP8266 microcontroller (optionally equipped with a WiFi or Bluetooth interface), or the like, in communication with said sensor and preferably configured for controlling the operation of the device 30 on the basis of the ozone concentration upstream of the catalytic filter 6. In this manner, it is possible to primarily control the revolution speed of the electric motor 2 according to a closed-loop speed control configuration, even stopping the electric motor 2 when the sensor 10 detects an output ozone concentration which is higher than the desired one. Moreover, said control unit is configured for executing the following operations:
detecting the signal produced by said sensor 10;
determining, on the basis of said signal, a quantity of ozone processed by said catalytic filter 6. This makes it possible to estimate a remaining life of said catalytic filter 6; in fact, said remaining life is inversely proportional to the quantity of ozone processed by said catalytic filter 6;
transmitting, via communication means comprised in said control unit, an alert message when the quantity of ozone to be processed exceeds a first threshold value.

This allows the user of the device 30 to advantageously keep the abating means 37 as efficient as possible. In this manner, it is possible to increase the quantity of ozone used for sanitizing the air in an inhabited environment without producing harmful ozone concentrations therein.

The sensor 10 preferably comprises a photoreceiver (e.g., a photoresistor) sensitive to C-type ultraviolet (UVC) rays. This allows exploiting the UVC lamps 8,9 already included in the device 30; in fact, the luminous intensity reading of the photoreceiver depends (according to the Lambert-Beer law) on the absorption of UVC rays by the ozone contained in the air flow F.

It is important to note that, as already specified, according to the present invention the UVC lamps 8,9 perform a dual function:
sterilizing any pathogenic elements that may still be present in the air flow F after the ozone treatment,
abating the ozone contained in the air flow F exiting the reaction chamber 36.

In this latter respect, should ozone production be considered, in certain operating conditions, to be excessive (because the quantity of ozone in the output flow exceeds the desired threshold), then the ozone production process could be stopped by turning off the ozone generator 35, while leaving the rest of the device 30 in operation and sanitizing the air only by means of the UVC lamps 8,9. In other words, at least one of the UVC lamps 8,9 is configured for emitting C-type ultraviolet light independently of the operating condition of the ozone generator 35.

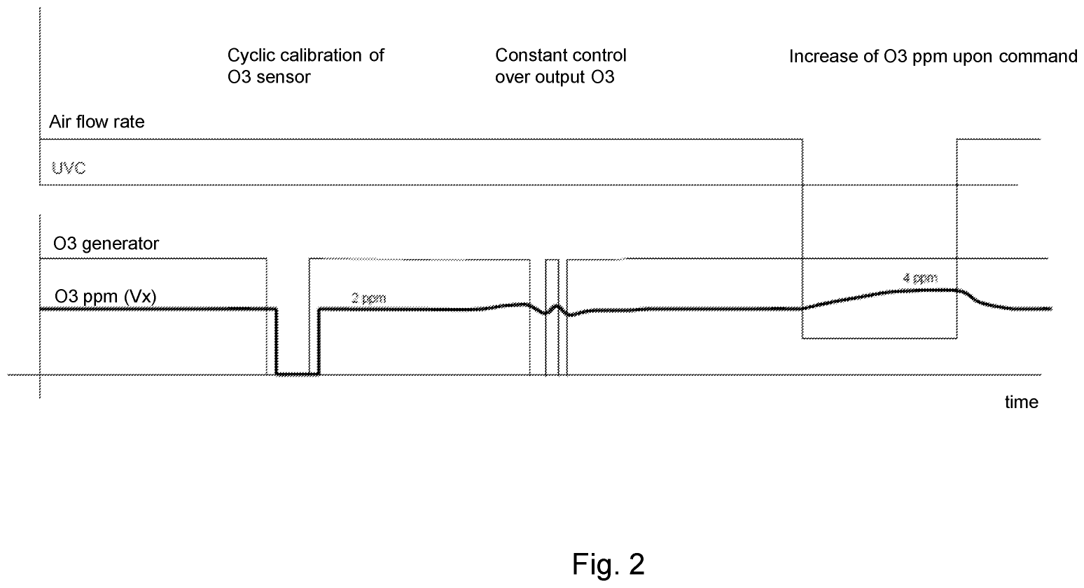

Also with reference to FIG. 2, the following will describe how the control unit of the device 30 executes a procedure for calibrating the sensor 10, so as to compensate for the brightness reduction undergone by the UVC lamps 8,9 due to ageing or dust. This will ensure accurate measurements throughout the life cycle of the abating means 37 and of the sensor 10.

More in detail, the control unit may be configured for executing a calibration method that comprises the following steps:
deactivating the ozone generator 35;
activating the UVC lamps 8,9 and the air flow generator 34 (in order to clean the inside of the device 30 from any ozone residues);
detecting the signal emitted by the sensor 10, generating a sample associated with ozone absence;
generating calibration data on the basis of the sample associated with ozone absence, wherein said calibration data comprise, for example, a value of a voltage produced by the sensor when it detects no ozone concentration.

Afterwards, it is possible to activate the ozone generator 35 again and determine the quantity of ozone processed by said catalytic filter 6 not only on the basis of the detected signal, but also on the basis of said calibration data.

The rate of the air flow F being known, it is possible to calculate an estimate of the quantity of ozone processed by the catalytic filter 6 which is more accurate because it is less affected by zero errors (also known as “offset errors”) caused by ageing of the sensor 10, so that the user of the device 30 can fully exploit the catalytic filter 6, as well as all the other abating means 37, throughout their service life. It is thus possible to increase the quantity of ozone used for sanitizing an inhabited environment without producing harmful ozone concentrations in such inhabited environment, because the utilization of the catalytic filter 6 can be kept under control.

It must also be pointed out that, when the ozone generator 35 is off, but the UVC lamps 8,9 and the air flow generator 34 are on, the device 30 can advantageously disinfect its own inside, i.e., the hollow body 31, the catalytic filter 6, etc., and, most importantly, abate any excess ozone that may be present in the environment.

Of course, the example described so far may be subject to many variations.

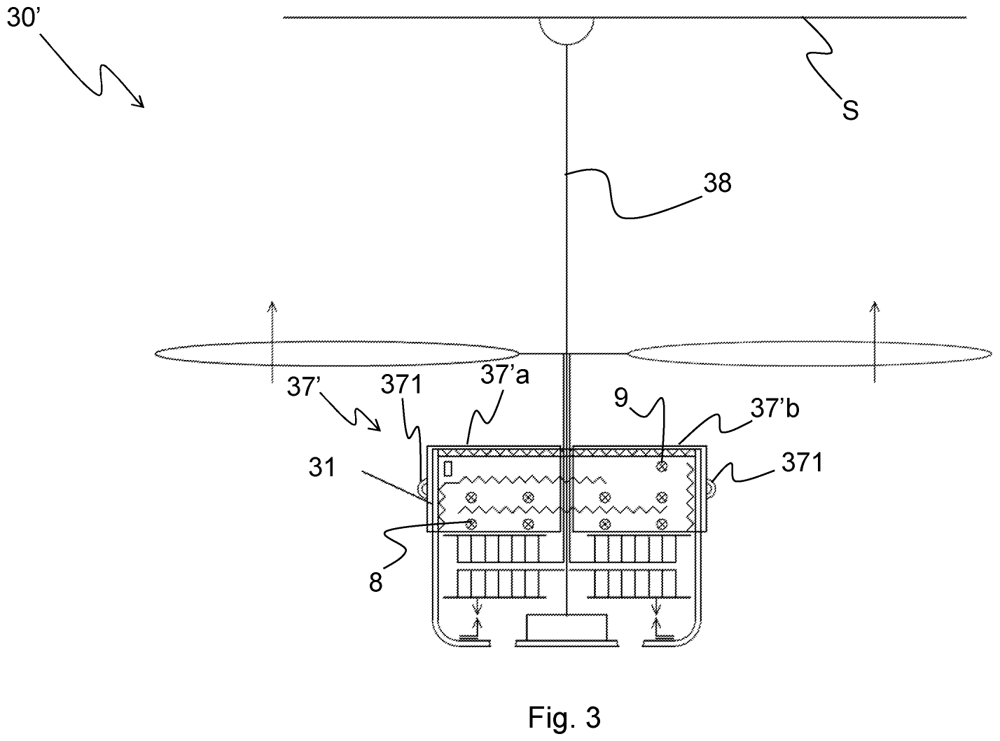

A first variant is shown in FIG. 3; for simplicity, the following description will only highlight those parts which make this and the next variants different from the above-described main embodiment; for the same reason, wherever possible the same reference numerals, with the addition of one or more apostrophes or Roman numerals, will be used for indicating structurally or functionally equivalent elements.

With reference to FIG. 3, the following will describe a first variant of the invention. This first variant comprises a device 30′ for treating and sanitizing air in an inhabited environment which is similar to the above-described device 30; such device 30′ comprises abating means 37′, which differ from those of the preceding embodiment in that they can be separated from the rest of the device (i.e., from the hollow body 31), so that the abating means 37′ (in particular the UVC lamps 8,9) can be maintained, in particular by cleaning/replacing the catalytic filter 6 and removing the dust that tends to accumulate within said abating means 37′. In fact, dust reduces the performance of the catalytic filter 6 and, most importantly, of the UVC lamps 8,9 and of the mirrors 51, since the light emitted and reflected by them is partly absorbed by dust particles without reaching the ozone molecules, thus reducing the effectiveness of the abating systems.

More in detail, the abating means 37′ comprise at least a first portion 37′a and a second portion 37′b, each one (independently) separable from the hollow body 31, so that they can advantageously be dismounted without unthreading the fixing cable or rod 38, i.e., without disconnecting the device 30′ from the ceiling S.

In order to facilitate the task of removing the abating means 37′ from the hollow body 31, each portion 37′a,37′b comprises a handle 371 that makes it easier for an operator to separate one of the portions from said hollow body 1 and service the device 30′. To make this possible, the shape of each portion 37′a,37′b is such that it is hooked to the hollow body 1 and held in position by gravity; in addition, each portion 37′a,37′b comprises electric contacts (not shown in the drawings), preferably of the sliding type, which make it possible to supply power to the UVC lamps 8,9 without hindering the separation of said portion 37′a,37′b from the hollow body 1.

Because of this, the abating means 37′ (in particular, the UVC lamps 8,9) can be subjected to frequent maintenance, so as to always remain fully functional. It is thus possible to use a large quantity of ozone in the device 30 for sanitizing an inhabited environment without producing harmful ozone concentrations therein.

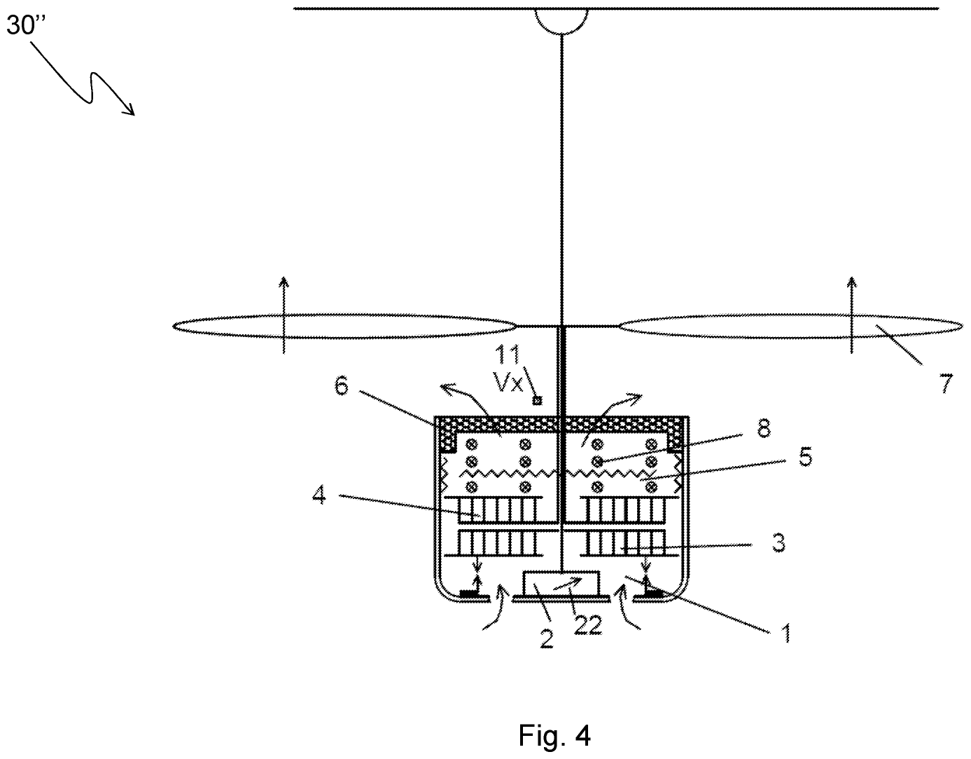

With reference to FIG. 4, the following will describe a second variant of the invention. This second variant comprises a device 30″ for treating and sanitizing air in an inhabited environment which is similar to the above-described devices 30,30′.

The device 30″ comprises a second sensor 11 capable of detecting an ozone concentration (e.g., an MQ131 sensor or the like), and the control unit is configured for controlling the operation of the device 30 on the basis of said ozone concentration downstream of the catalytic filter 6; in fact, such sensor 11 is positioned outside the hollow body 31, i.e., downstream of the catalytic filter 6, and can produce a signal representing an ozone concentration external to the hollow body 31. This second sensor 11 is in communication with the control unit of the device 30″ via a wired connection or a radio (wireless) connection, e.g., via Bluetooth or WiFi.

To this end, the control unit may be configured for executing the following operations:
receiving, via the communication means, an ozone concentration datum read by said second sensor 11;
activating or deactivating (or slowing down) the ozone generator 35 on the basis of said ozone concentration datum; for example, if the ozone concentration detected by the second sensor 11 exceeds 0.1 parts per million (ppm), i.e., 100 parts per billion (ppb), the ozone generator 35 will be turned off, otherwise it will be turned or kept on.

It must be pointed out that the second sensor 11 may also be comprised in a remote control or a mobile device, e.g., a smartphone, which may also be configured for displaying a current value of the output ozone concentration.

The presence of the second sensor 11 is useful to prevent the inhabitants of an environment in which a device 30″ has been installed from being subjected to excessively high ozone concentrations, thus inhaling excessive quantities of ozone. It is thus possible to increase the quantity of ozone used in the device 30 for sanitizing an inhabited environment without producing harmful ozone concentrations therein.

In this case as well, even when the ozone generator 35 is stopped or slowed down, the rest of the device 30″ can be left in operation to sanitize the air only by means of the UVC lamps 8,9.

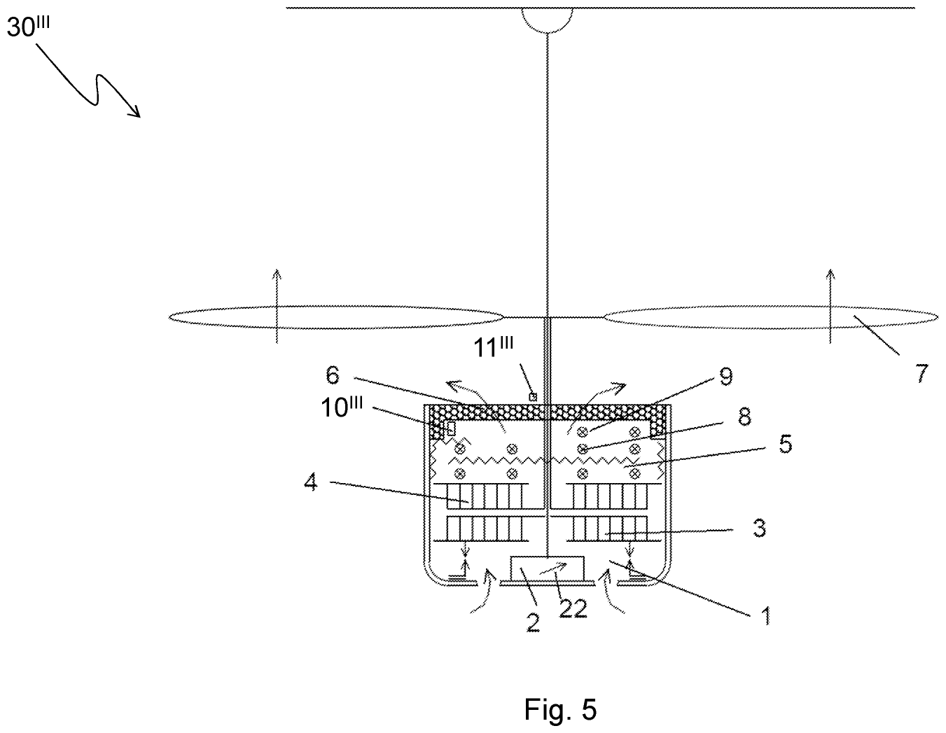

With reference to FIG. 5, the following will describe a third variant of the invention. This third variant comprises a device 30^{III} for treating and sanitizing air in an inhabited environment which is similar to the above-described devices 30,30′,30″.

The device 30^{III} comprises a first sensor 10^{III} and a second sensor 11^{III}, wherein said first sensor 10^{III} is similar to the sensor 10 of the device 30 and is positioned inside the hollow body 31, while said second sensor 11^{III} is similar to the sensor 11 of the preceding variant and is likewise positioned outside said hollow body 31.

The presence of the two sensors 10^{III},11^{III} permits a more accurate monitoring of the operation of the catalytic filter 6, leading to immediate recognition of any malfunction or reduced effectiveness thereof.

To this end, the control unit may be configured for executing the following operations:
detecting, through said first sensor 10III, a first ozone concentration datum;
detecting, through said second sensor 11III, a second ozone concentration datum;
determining a remaining life datum representing a remaining life of the catalytic filter 6 (expressed in working hours, ozone amount that can still be processed, or the like) on the basis of the first ozone concentration datum (coming from said first sensor 10III) and second ozone concentration datum (coming from said second sensor 11III), e.g., by evaluating the difference between the two concentration data and comparing the result with a calibration data set previously determined by means of laboratory tests;
transmitting, via the communication means, said remaining life datum to a display device, preferably a mobile terminal.

It must be highlighted that the control unit may be configured for inhibiting the operation of the ozone generator 35 if it determines that the remaining life of the catalytic filter 6 is equal to zero or if the output ozone values tend to exceed a (safety) threshold value.

In both cases, even when the ozone generator 35 is turned off, the rest of the device 30′″ can be left in operation to sanitize the air only by means of the UVC lamps 8,9.

Thus, the catalytic filter 6 can be replaced when actually necessary, since its efficiency is constantly monitored. It is therefore possible to use a large quantity of ozone for sanitizing the air in an inhabited environment, without producing harmful ozone concentrations therein.

Furthermore, in comparison with the above-described embodiments, this variant permits reducing the risk of false alarms (leading to inhibition of the ozone generator 35), which might be produced, for example, by an ozone generator located in the same environment and external to the device 30^{III}, e.g., a water sanitizer or the like, or when a summer storm is coming, etc.

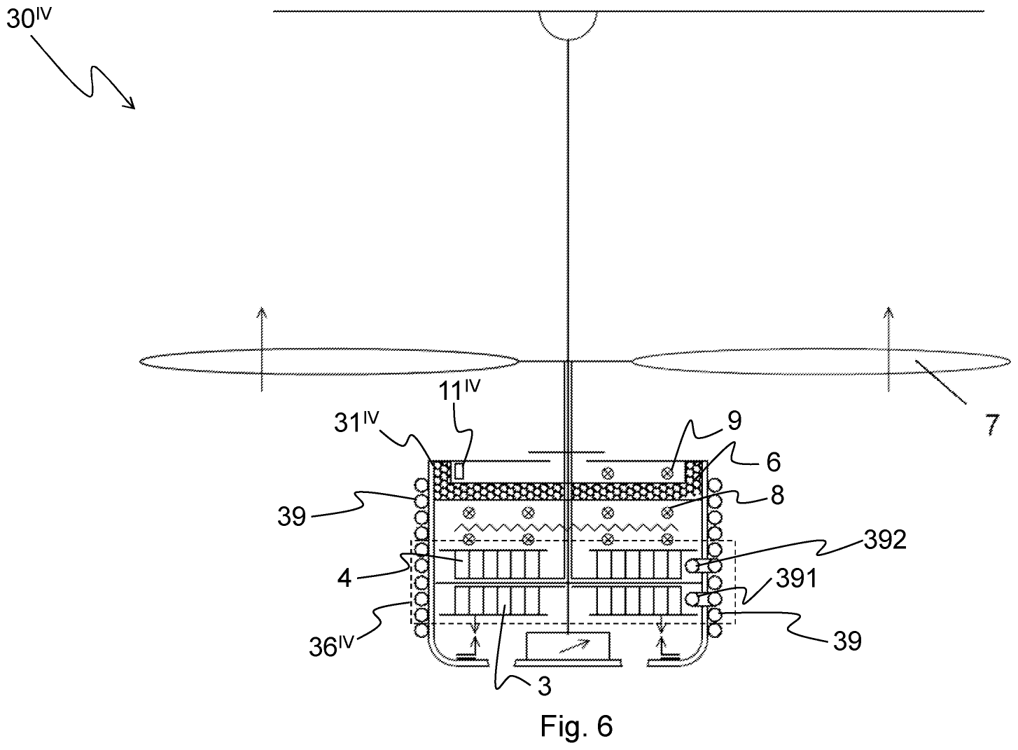

With reference to FIG. 6, the following will describe a fourth variant of the invention. This fourth variant comprises a device 30^{IV} for treating and sanitizing air in an inhabited environment which is similar to the above-described devices 30, 30′, 30″, 30^{III}.

The device 30^{IV} comprises a second sensor 11^{IV} similar to the sensor 10 comprised in the first embodiment, i.e., the second sensor 11^{IV} preferably comprises a photoreceiver (e.g., a photoresistor) sensitive to C-type ultraviolet (UVC) rays; such sensor 11^{IV} is positioned inside the hollow body 31, downstream of the catalytic filter 6, and can produce a signal representing an ozone concentration immediately downstream of the catalytic filter 6. Such a position makes it advantageously possible to exploit the light produced by the UVC lamps 9 (which in this variant are also positioned downstream of the catalytic filter 6) in order to sense the ozone concentration without requiring the use of a sensor like the second sensor 11 of the preceding variant, i.e., an MQ131 sensor, which uses tin dioxide and therefore, in addition to being sensitive to ozone, is also sensitive to chlorine (Cl₂) and nitrogen dioxide (NO₂). This makes it less likely that the ozone generator 35 will be inhibited when cleaning activities are carried out by using sodium hypochlorite in the environment where the device 30^{IV} has been installed.

In order to prevent the UVC lamps 9 from diffusing ultraviolet light in the inhabited environment, the device 30^{IV} comprises a hollow body 31^{IV} which is similar to that of the previously described embodiments, while however having a second opening so shaped as to prevent, or anyway strongly limit, the exit of ultraviolet light, e.g., a siphon-like shape or the like.

It is thus possible to use a large quantity of ozone for sanitizing an inhabited environment without producing harmful ozone concentrations therein.

In combination with or as an alternative to the above, the device 30^{IV} preferably comprises a reaction chamber 36^{IV} similar to the reaction chamber 36 of the main embodiment; such reaction chamber 36^{IV} is divided into a first section, in which the intake rotor 3 (i.e., the air flow generator) is located, and a second section, in which the turbine 4 is located, these two sections being mutually connected by means of a tube 39 comprised in said device 30^{IV} and preferably wound multiple times around the hollow body 31^{IV} (i.e., it has a serpentine shape), thus extending the path that the air flow F has to travel inside the device 30^{IV}. The tube 39 preferably comprises an inlet 391 located in proximity to the intake rotor 3 and an outlet 392 located in proximity to the turbine 4. With such an arrangement, the tube 39 can pick up the pressure waves generated by the blades of the intake rotor 3 and of the turbine 4 during their rotation, thus making it possible to exploit these rapid and frequent pressure variations and the pressure increase to cause the ozone in the air flow F to interact within the tube 39 and sanitize the air flow F more effectively.

It is thus possible to increase the quantity of ozone used for sanitizing an inhabited environment, without producing harmful ozone concentrations therein.

Moreover, the serpentine shape of the tube 39 acts as a radiator; in fact, said tube 39 can cool the compressed mixture of air and ozone from a temperature of, preferably, 80° C. to a temperature of, preferably, 40° C.

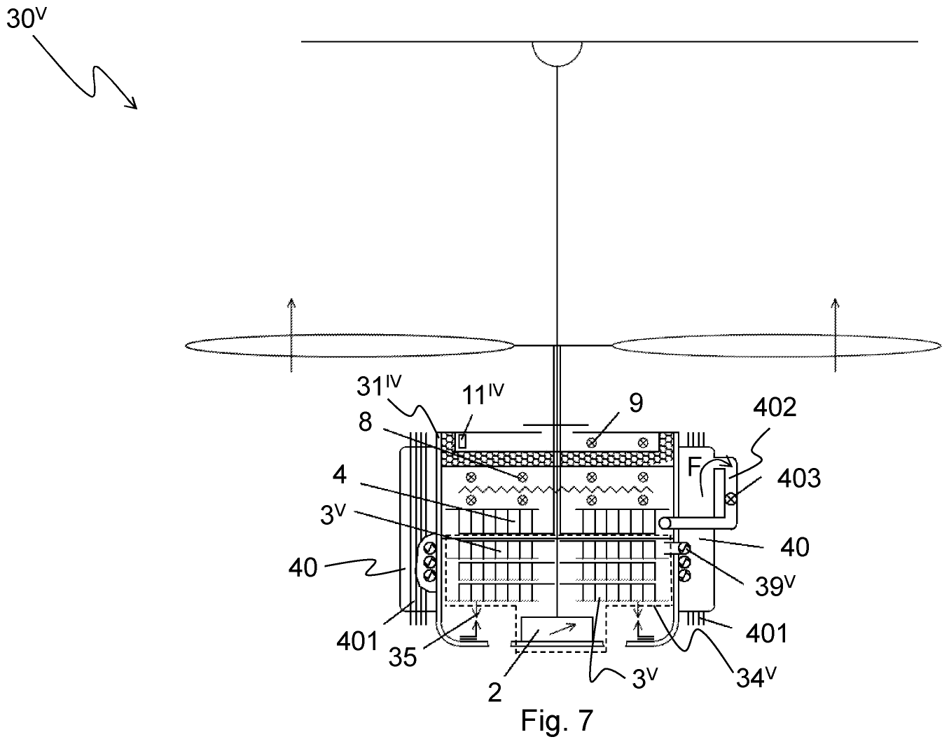

With reference to FIG. 7, the following will describe a fifth variant of the invention. This fifth variant comprises a device 30^{V} for treating and sanitizing the air in an inhabited environment which is similar to the above-described devices 30, 30′, 30″, 30^{III}, 30^{IV.}

The device 30^{V} comprises the following elements:
an air tank 40 under a pressure higher than the atmospheric pressure, preferably having an annular shape surrounding the hollow body 31IV;
a tube 39V similar to the tube 39 of the preceding embodiment, i.e., preferably wound multiple times around the hollow body 31IV and, preferably, internally comprising one or more turbinator elements (e.g., fixed fins within said tube 39V) capable of increasing the contact between ozone and air, wherein said tube 39V puts the air flow generator 34 in communication with the tank 40, which also comprises a drain duct 402 in communication with the turbine 4;
a valve 403, preferably actuated by an actuator and positioned along said drain duct 402, which can adjust the expansion of the compressed and cooled fluid which is supplied into the turbine 4 and which provides the energy necessary for driving the blades.

It must also be pointed out that the air flow generator 34 of this embodiment preferably comprises a multi-stage intake rotor 3^{V}, i.e., a rotor capable of producing a higher pressure difference than the rotor 3 of the first embodiment.

When the device 30^{V} is in an operating condition, the air of the flow F is kept within the tank 40 at a pressure above the atmospheric pressure for a longer time than according to the preceding variants of the invention; in addition, this variant provides a pressure jump across the valve 403 which is greater than the one provided by any other variant previously described. This pressure jump permits accelerating the reaction process of the ozone contained in the air flow F within the tube 39^{V}, thus more effectively sanitizing the air flow F.

It is thus possible to increase the quantity of ozone used within the device 30^{V} for sanitizing an inhabited environment, without producing harmful ozone concentrations therein.

In addition, the valve 403 may also comprise an actuator (not shown in the drawings) capable of actuating said valve 403, wherein the control unit controls said actuator.

This allows the control unit to control the rate of flow F through the device 30^{V}, so as to slow down the feeding of the air flow F into the turbine 4, and hence into the environment where the device 30^{V} has been installed, should situations arise wherein the sensor 11^{IV} detects an excessively high ozone value.

Furthermore, the control unit may be configured for controlling the valve 403 in such a way as to either open or close the valve 403 at such a frequency as to trigger a resonance phenomenon in the tank 40 which, through continual overpressures and decompressions, will contribute to accelerate the reaction process of the ozone contained in the air inside the tank 40, thus sanitizing the air flow F more effectively.

It is thus possible to increase the quantity of ozone used within the device 30^{V} for sanitizing the air in an inhabited environment, without producing harmful ozone concentrations therein.

The tank 40 preferably comprises also cooling fins 401 that contribute to lower the temperature of the air in the tank 40, which, following the compression process effected by the intake rotor 3^{V}, has a temperature higher than room temperature, while downstream of the expansion valve 403 the temperature of the ozone-enriched air decreases during the expansion and then decreases even more during the isentropic expansion in the turbine 4, so that its relative humidity is advantageously increased in comparison with the atmospheric relative humidity, even until saturation with fog production, resulting in higher sterilization power and greater ozone abatement before the ozone is processed by the UVC lamps 8,9 and by the catalytic filter 6.

It is thus possible to increase the quantity of ozone used within the device 30^{V} for sanitizing the air in an inhabited environment, without producing harmful ozone concentrations therein.

Moreover, the presence of the valve 403 is also useful to increase the time of contact between the ozone and the air to be treated, because it is possible to completely close said valve 403 while keeping the motor 2 in operation, preferably executing 15-minute cycles. In this manner, a quantity of pressurized air, equal to the volume of the chamber 40, can be cyclically kept in contact with the ozone for a longer time, so as to produce a more complete sterilizing effect and a better sanitization of the device 30^{V.}

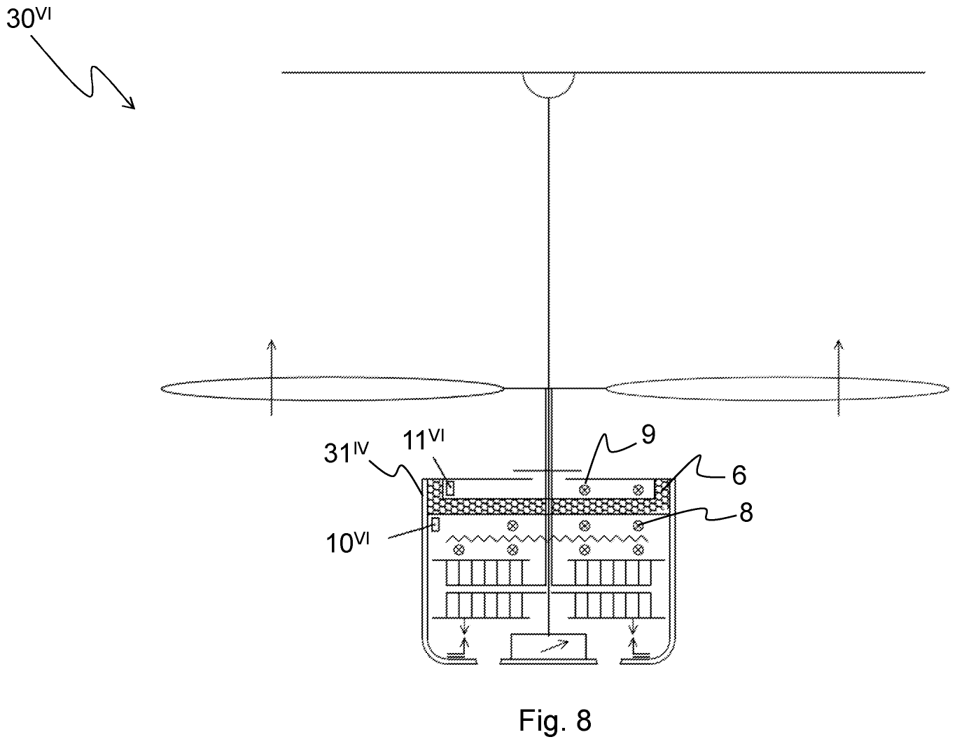

With reference to FIG. 8, the following will describe a sixth variant of the invention. This sixth variant comprises a device 30^{VI} for treating and sanitizing air in an inhabited environment which is similar to the above-described devices 30, 30′, 30″, 30^{III}, 30^{IV}, 30^{V.}

The device 30^{VI} comprises the following elements:
a first sensor 10^{VI} similar to the sensor 10 of the device 30 and positioned inside the hollow body 31^{IV};
a second sensor 11^{VI} similar to the second sensor 11^{IV} of the device 30^{IV} and positioned inside the hollow body 31 downstream of the catalytic filter 6.

Since each one of the sensors 10^{VI}, 11^{VI} comprises a photoreceiver (e.g., a photoresistor) sensitive to C-type ultraviolet (UVC) rays and is positioned in proximity to the UVC lamps 8,9, it is advantageously possible to exploit the light produced by said UVC lamps 8,9 to detect the ozone concentration without having to use a sensor like the second sensor 11 of the preceding variant, i.e., like the MQ131 sensor, which uses tin dioxide and therefore, in addition to being sensitive to ozone, is also sensitive to chlorine (Cl₂) and nitrogen dioxide (NO₂).

It is thus less likely that the ozone generator 35 will be inhibited when cleaning activities are carried out in the domestic environment by using sodium hypochlorite. Furthermore, it is also possible to calibrate both sensors 10^{VI}, 11^{VI} by using the calibration procedure described for the main embodiment.

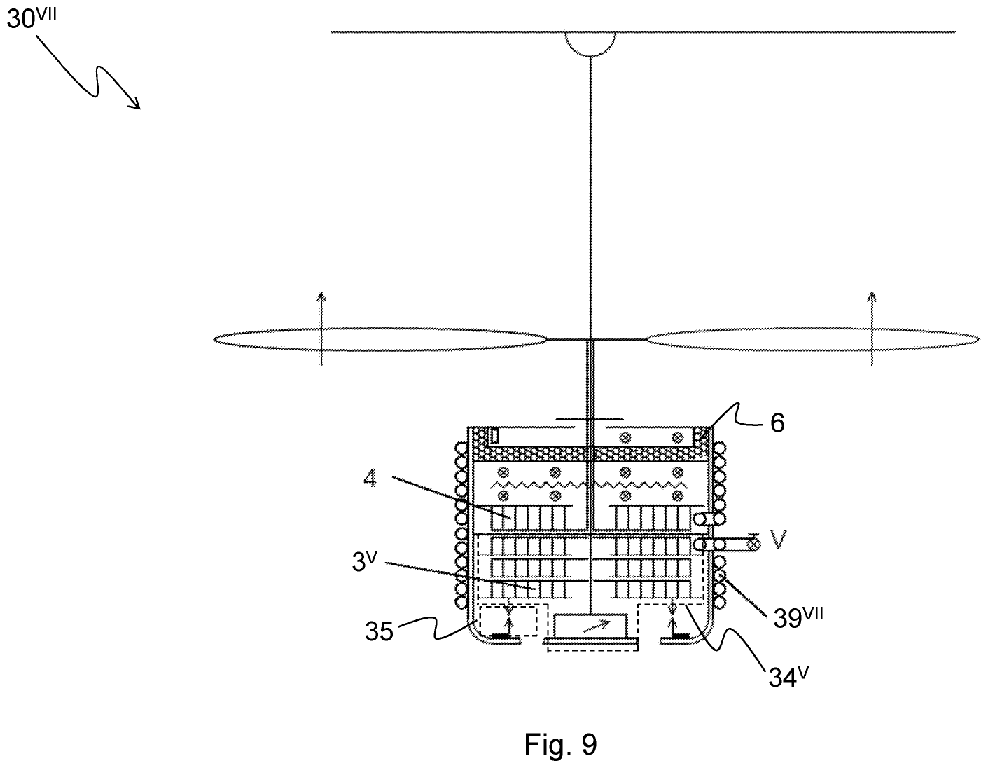

With reference to FIG. 9, the following will describe a seventh variant of the invention. This seventh variant comprises a device 30^{VII} according to the invention which is similar to the above-described device 30^{IV.}

The device 30^{VII} comprises the following elements:
a tube 39^{VII} similar to the tube 39 of the device 30^{IV;}
a valve V comprising an inlet in communication with the inside of the tube 39^{VII} and an outlet in communication with the outside of the device 30^{VII}, i.e., with the environment outside said device 30^{VII}.

Therefore, when said valve V is in the open position, the air flow will be discharged into the atmosphere immediately after having passed through the ozone generator 35 and the air flow generator 34^{V}; when the valve V is in the closed position, the device 30^{VII} will operate like the device 30^{IV}.

The valve V preferably comprises an actuator in communication with a control unit similar to the one described for the preceding variants; such control unit is preferably configured for executing the following steps:
receiving, via the communication means, an activation message, preferably transmitted by a mobile terminal controlled by a user of the device 30^{VII};
generating one or more signals capable of activating the ozone generator 35, the air flow generator 34^{V}, and the actuator of the valve V to open said valve V.

This makes it possible to concentrate large quantities of ozone in a room in the absence of people, so as to preserve (i.e., avoid consuming) the catalytic filter 6. This increases the service life of the catalytic filter 6, since it is crossed by ozone-enriched air only when people are present in the room or shortly before they enter said room.

In fact, the control unit is also configured for executing the following steps:
receiving, via the communication means, a deactivation message, preferably transmitted by a mobile terminal controlled by the user of the device 30^{VII};
generating one or more signals capable of activating the ozone generator 35, the air flow generator 34^{V}, and the actuator of the valve V to close said valve V.

In this manner, the catalytic filter 6 can remain efficient for a longer time, thus allowing the use of a larger quantity of ozone within the device 30^{VII} for sanitizing the air in an environment when it is inhabited without producing harmful ozone concentrations in said environment.

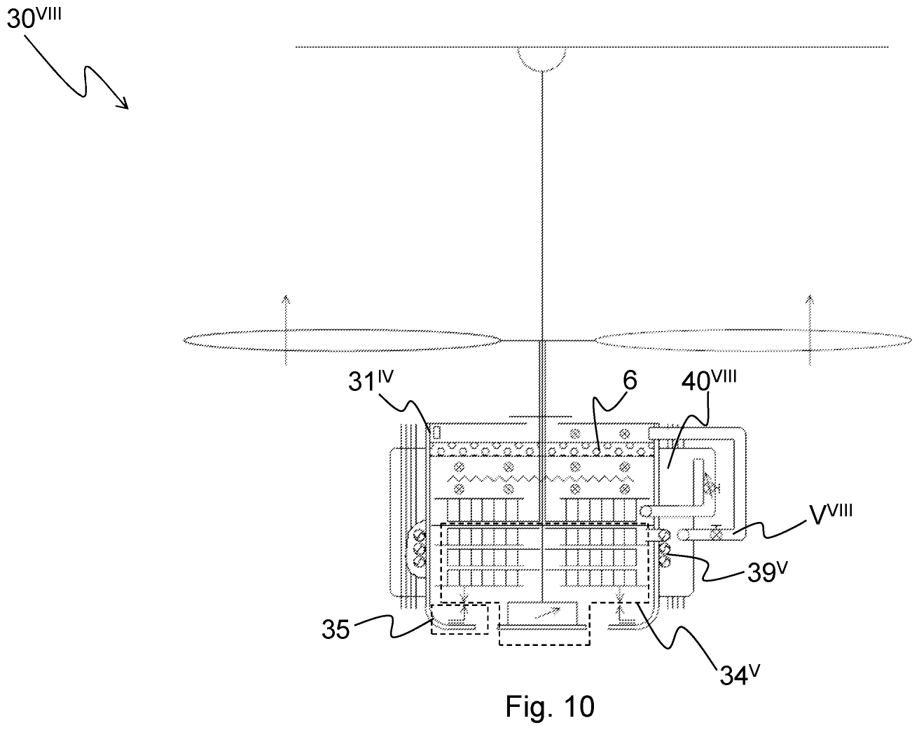

With reference to FIG. 10, the following will describe an eighth variant of the invention. This eighth variant comprises a device 30^{VIII} according to the invention which is similar to the above-described device 30^{V.}

The device 30^{VIII} comprises the following elements:
a tank 40^{VIII} similar to the tank 40 of the device 30^{V;}
a valve V^{VIII} similar to the valve V of the previously described embodiment, and comprising an inlet in communication with the inside of the tank 40^{VIII} (and therefore also with the inside of the tube 39^{V}) and an outlet in communication with the space of the hollow body 31^{IV} downstream of the catalytic filter 6 or with the outside of the device 3^{VIII}.

Therefore, when said valve V^{VIII} is in the open position, the air flow will be discharged into the atmosphere after having passed through the ozone generator 35 and the air flow generator 34^{V}; when the valve V^{VIII} is in the closed position, the device 30^{VIII} will operate like the device 30^{V}.

The operation of the device 30^{VIII} is similar to that of the device 30^{VIII}, from which it differs for a different position of the valve V^{VIII}, which makes it possible to preserve the catalytic filter 6 as already described with reference to the preceding embodiment of the invention.

In this manner, the catalytic filter 6 can remain efficient for a longer time, thus allowing the use of a larger quantity of ozone for sanitizing an environment when it is inhabited without producing harmful ozone concentrations in said environment.

In addition to the above-described variants, the invention may also be implemented as a piece of furniture to be laid on a surface or fastened to a wall by means of suitable supports. This solution overcomes any problems related to the weight of the equipment, and also allows enhancing the paths of the air to be sanitized and the positioning of the abating means, so as to more effectively abate the ozone after it has performed its air sanitizing action.

Some of the possible variants of the invention have been described above, but it will be clear to those skilled in the art that other embodiments may also be implemented in practice, wherein several elements may be replaced with other technically equivalent elements. The present invention is not, therefore, limited to the above-described illustrative examples, but may be subject to various modifications, improvements, replacements of equivalent parts and elements without however departing from the basic inventive idea, as specified in the following claims.

1.-18. (canceled)

19. A device for treating and sanitizing air in an inhabited environment, comprising:
a hollow body comprising a first opening for the entrance of an air flow and a second opening for the exit of said air flow;
an air flow generator configured for producing said air flow;
an ozone generator configured for adding ozone to said air flow within said hollow body;
a reaction chamber positioned inside said hollow body downstream of the ozone generator, wherein said reaction chamber is configured for changing one or more physical characteristics of said air flow within said hollow body, wherein said reaction chamber comprises means for compressing and expanding said air flow F within said hollow body, and wherein said characteristics include pressure, temperature, enthalpy, velocity, turbulence, dew point; and
abating means configured for abating the ozone contained in the air flow exiting said reaction chamber.

20. The device according to claim 19, wherein the reaction chamber is configured through appropriate shaping, for changing one or more of said physical characteristics.

21. The device according to claim 19, wherein the abating means comprise a catalytic filter.

22. The device according to claim 21, further comprising a first sensor, positioned upstream of the catalytic filter and capable of detecting a first ozone concentration, and/or a second sensor, positioned downstream of said catalytic filter and capable of detecting a second ozone concentration.

23. The device according to claim 22, further comprising a control unit in communication with said first sensor and/or said second sensor, and configured for controlling the operation of said device on the basis of said first ozone concentration and/or said second ozone concentration.

24. The device according to claim 23, wherein the control unit comprises communication means, is in communication with said first sensor, and is configured for:
detecting a signal produced by said first sensor;
determining, on the basis of said signal, a quantity of ozone to be processed by said catalytic filter; and
transmitting, via the communication means, an alert message when the quantity of ozone to be processed exceeds a first threshold value.

25. The device according to claim 19, wherein the abating means comprise at least one lamp capable of emitting C-type ultraviolet light.

26. The device according to claim 25, wherein the lamp is configured for emitting C-type ultraviolet light independently of the operating condition of the ozone generator.

27. The device according to the claim 23, wherein said first sensor and/or said second sensor comprise a photoreceiver sensitive to C-type ultraviolet rays.

28. The device according to claim 24, wherein the control unit is configured for:
deactivating the ozone generator;
activating said at least one lamp capable of emitting C-type ultraviolet light and the air flow generator;
detecting the signal emitted by said first sensor or said second sensor, generating a sample associated with ozone absence;
generating calibration data on the basis of the sample associated with ozone absence; and wherein
the control unit is configured for determining the quantity of ozone to be processed by said catalytic filter also on the basis of said calibration data.

29. The device according to claim 19, wherein the abating means comprise at least a first portion and a second portion, and wherein each one of said portions can be separated from the hollow body.

30. The device according to claim 19, wherein the reaction chamber comprises a turbine configured to be actuated by the air flow generated by the air flow generator.

31. The device according to claim 30, further comprising a propeller coupled to said turbine and positioned outside the hollow body.

32. The device according to claim 30, wherein the reaction chamber is divided into a first section, in which the air flow generator is located, and a second section, in which the turbine is located, wherein said device comprises a tube that mutually connects said first section and said second section of the reaction chamber.

33. The device according to claim 32, comprising
a tank which can contain air at a pressure higher than an atmospheric pressure, and which is positioned along the tube, wherein said tube comprises a drain duct in communication with the tank and the turbine; and
a valve positioned along said drain duct.

34. The device according to claim 33, wherein the valve comprises an actuator capable of actuating said valve, and a control unit is configured for controlling said actuator.

35. The device according to claim 33, wherein the tank comprises one or more cooling fins.

36. The device according to claim 32, wherein the tube comprises one or more turbinators positioned inside said tube.

37. The device according to claim 32, comprising a valve that comprises an inlet in communication with the tube and an outlet in communication with said environment.
